(19) United States
(12) Patent Application Publication
(10) Pub. No.: US 2010/0282594 A1
(43) Pub. Date: Nov. 11, 2010

(54) METHOD AND DEVICE FOR CLEANING A HOT AIR STREAM

(76) Inventor: Manfred H. Langner, Frankfurt/Main (DE)

Correspondence Address: WRB-IP LLP, 801 N. Pitt Sreet , Suite 123, ALEXANDRIA, VA, 22314, US

(21) Appl. No.: 12/680,967

(22) PCT Filed: Sep. 24, 2008
(86) PCT No.: PCT/EP08/08064

§ 371 Date: May 13, 2010

(30) Foreign Application Priority Data
Oct. 2, 2007 (DE) 10 2007 047 356.9

(51) Int. Cl. B01D 53/72; A61L 9/22; B01J 12/00; B01D 50/00

(52) U.S. Cl. 204/157.3; 422/186; 422/121

(57) ABSTRACT

A method is provided for cleaning a hot air stream, wherein the air stream is conducted through a cooling device and then through a filter device, which includes at least one plasma production device for removing gaseous organic carbon compounds. The cooling device cools the hot air stream to temperatures below 60° C. The air stream to be cleaned is accelerated before entering the filter device and is deflected by means of suitable guiding surfaces. A cleaning device is provided for a hot air stream and includes a filter device with a plasma production device for removing gaseous organic carbon compounds and a cooling device connected upstream of the filter device. The cooling device includes a spraying device for a coolant. The filter device includes an open-pored hydrophilic foam material, the plasma production device and a suitable absorber material, through which the air stream flows consecutively.

### BACKGROUND AND SUMMARY

The invention relates to a method for cleaning a hot air stream.

Different methods are known that can be used advantageously for cleaning an air stream. Cleaning an air stream not only comprises filtering out undesirable impurities but also separating, breaking out or destroying or converting particles, aerosols, condensate, moisture and especially gaseous organic carbon and hydrocarbon compounds.

Filtering out or breaking down gaseous organic carbon or hydrocarbon compounds is especially practical and desirable when the air stream contains unpleasant odors or when bothersome odor is to be avoided following the cleaning of the air stream.

Methods are known in which small particles and condensate particles are filtered out of and separated from the air stream by means of, for example, mechanical impingement filters or suitable absorption materials, such as active carbon.

If the air stream to be cleaned is a hot air stream, such as those exiting from a hot air steamer, the air stream must be sufficiently cooled off before it comes in contact with the temperature sensitive active carbon. In this context, a hot air stream is an air stream whose temperature ranges considerably above room temperature and reaches, for example, 150° C. or even in excess of 250° C. A hot air stream escapes, for example, when opening the doors of a hot air steamer that is used for private or industrial food preparation.

To ensure that the hot air stream is sufficiently cooled, it is known to either guide the air stream along a sufficiently long delay line up to the active carbon so that the air stream can be sufficiently cooled or to guide the air stream through a cooling device.

Other cleaning methods are known in which a cleaning effect can be obtained that is mostly or completely independent of the air stream temperature. One example of a temperature-independent cleaning of an air stream are the known plasma cleaning methods in which a plasma is produced in the air stream by means of a suitable plasma production device with the plasma effectively breaking down gaseous organic carbon compounds, among other things, and neutralizing odor nuisances caused by these carbon compounds.

It is desirable to develop a method for cleaning a hot air stream in a manner that ensures that it is possible to achieve a very effective cleaning effect in the shortest possible time and as cost-effectively as possible.

According to an aspect of the present invention, a method is provided for cleaning a hot air stream, wherein the air stream is guided through a cooling device and then through a filter device, which comprises at least one plasma production device for removing gaseous organic carbon compounds. Preferably it is a multi-stage filter device in which different cleaning methods such as mechanical cleaning, absorption or electrostatic filtering methods are used. Studies have shown that a combination of the method steps according to an aspect of the invention allows for surprisingly efficient cleaning of and effective removal of odor from a hot air stream to be cleaned within a short period of time and with minimal space requirements. It also was found that the combination of a mostly temperature-independent cleaning method, such as that of plasma cleaning, with preceding cooling of the air stream can improve the cleaning method as well, in particular when apart from the plasma cleaning additional method steps for cleaning the air stream are provided or additional cleaning methods are used, respectively.

Preferably the hot air stream is cooled to temperatures below 60° C. by means of the cooling device before the cooled air stream is subsequently introduced into the filter device. While, for example, mechanical impingement filters can withstand higher temperatures, absorbent filter materials such as active carbon become increasingly less effective at temperatures above 50° C. to 60° C. and might even become ineffective or can be destroyed. By cooling the hot air stream to temperatures below 60° C. or even 50° C., one can ensure that as far as the temperature of the air stream is concerned, the order of the subsequently guided cleaning steps is no longer significant and the individual cleaning steps can be combined based solely with regard to the cleaning effect that can be obtained and can be performed in a given order.

According to one embodiment of the inventive thought, the air stream to be cleaned is accelerated before entering the filter device. It was found that accelerating the air stream to be cleaned supports an efficient capture of the air stream to be cleaned and introduction into the filter device. The fact that it might be inevitable that the cooling effect is reduced due to the acceleration of the air stream to be cleaned is of insignificant consequence in practical applications.

The effectiveness of individual cleaning method steps or the respective filter devices that are used can be improved by deflecting the air stream to be cleaned by means of suitable guiding surfaces. The guiding surfaces can either force a given reduction of the effective cross sectional area of the air stream or can cause an even distribution of the air stream to be cleaned across the active cross sectional area of a downstream filter device.

The invention also concerns, according to an aspect thereof, a cleaning device for a hot air stream that, according to an aspect of the invention, comprises a multi-stage filter device with at least one plasma production device for removing gaseous organic carbon compounds and a cooling device connected upstream of the filter device.

Preferably, the cooling device comprises a spraying device for a coolant. Especially when using a liquid coolant such as water or a different suitable coolant, the hot air stream can simultaneously be cooled and cleaned by washing out particles or condensate drops with the sprayed coolant and carrying them off by means of a collecting pit.

Instead of using a spraying device for a gaseous or liquid coolant, it is, of course, also possible to use a heat exchanger with cooled surfaces that can cause the hot air stream to cool without the use of a coolant.

In an especially advantageous embodiment of the inventive thought, the filter device comprises an open-pored hydrophilic foam material, the plasma production device and a suitable absorber material through which the air stream flows consecutively. It was found that by using a multi-stage filter device and especially by combining a mechanical filter and separation process like the one based on the open-pored hydrophilic foam material, with plasma cleaning and a subsequent filtering process by means of an absorber material, for example active coal, an especially efficient cleaning effect is obtained. In an especially advantageous manner the plasma production device forces a dielectric discharge in the air stream to be cleaned, by means of which the odors and especially gaseous organic carbon compounds can be destroyed or broken down and thus can be neutralized.

According to an advantageous embodiment of the inventive thought, the filter device additionally comprises an electric filter. The electric filters known from practice customarily comprise an ionization device and a collector; the particles and condensation drops in the air stream are electrically charged by means of the ionization device and separated at the collector surfaces that have the opposite electric charge.

In an especially advantageous manner the cleaning device comprises an air intake with a narrowing and subsequently widening intake opening. The narrowing intake opening forces the air stream that flows into the air intake to accelerate in a suction area upstream of the air intake which results in a substantially improved capture of the air to be cleaned. The subsequently widening intake opening causes the streaming air stream to decelerate so that larger particles and condensate drops that are taken along are separated based on the deceleration of the air stream alone.

In order to advantageously influence the flow properties of the air stream as it passes through the filter device, the cleaning device comprises a plurality of fans arranged side by side. These flow generation devices that are simply called fans influence the velocity of the air stream that is guided through the filter device. To account for an advantageous use of the available space, it also is feasible to guide the air stream around one or a plurality of corners or to change its direction so that additional fans are required to prevent a deceleration of the air stream, which would be undesirable. Since especially the use of a cleaning device according to an aspect of the invention with hot air steamers generates large amounts of hot air to be cleaned for short periods of time, with the hot air having to be drawn in as effectively as possible and having to be guided through the cooling device and the subsequent filter device, it was found to be practical to use a plurality of smaller fans arranged side by side instead of a single, large fan. The plurality of fans can be switched comparatively faster between a slow and a fast fan stage if this is required due to a sudden, large amount of hot air, which customarily happens when a hot air steamer is opened.

To be able to advantageously dictate the spatial distribution of the air stream to be cleaned while the air stream to be cleaned flows through the filter device, a plurality of guiding surfaces are arranged side by side in the direction of flow. The guiding surfaces provide simple means for causing or at least facilitating an even or a small cross-sectional area with reduced guided flow.

### BRIEF DESCRIPTION OF THE DRAWINGS

The exemplary embodiments shown in the drawing are explained in more detail below. The following is shown:

FIG. 1 shows a schematic sectional view of a cleaning device and

FIG. 2 shows a schematic sectional view of a different cleaning device.

### DETAILED DESCRIPTION

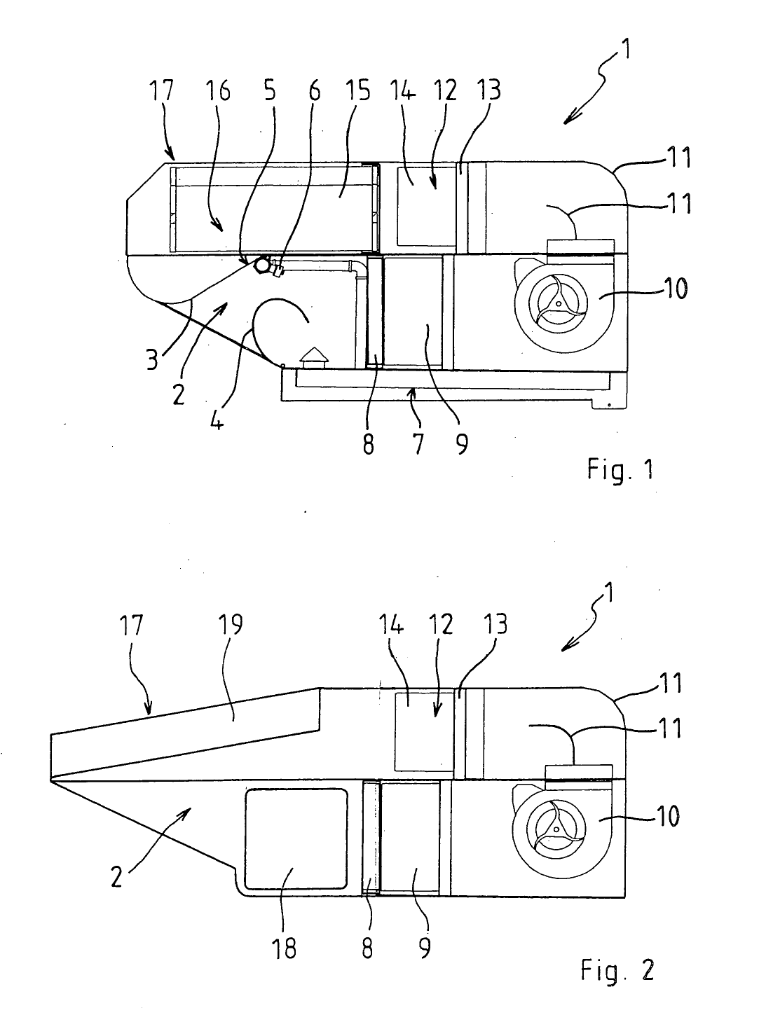

The schematically shown cleaning device I shown in FIG. 1 comprises a narrowing and subsequently widening intake opening 2 that is delimited by two curved stream guide impingement baffles 3, 4 arranged at a distance from one another. The drawn in air stream to be cleaned that enters through the intake opening 2 is accelerated by the narrowing cross section of the intake opening 2 so that the air to be cleaned is drawn in more effectively and the capture range of the cleaning device I is enlarged.

A spraying device 5 for a liquid coolant is arranged directly following the intake opening 2. The spraying device 5 comprises a plurality of spray jets 6 arranged in the direction of flow with which the liquid coolant is injected into and distributed in the air stream to be cleaned. A collecting pit 7 for collecting and subsequently removing the sprayed liquid coolant is arranged on the bottom side of the cleaning device. This is followed in the direction of flow by a completely covered layer of an open-pored hydrophilic foam material 8 that is available for the air stream. This open-pored, hydrophilic foam material 8 filters out solid particles and liquid drops from the air stream and removes them. In this context the open-pored hydrophilic foam material has a very advantageous property in that condensation drops and impurities are effectively filtered out of the air stream despite a low flow resistance and are removed from the open-pored hydrophilic foam material itself in a self-cleaning process and are moved to the collecting pit 7.

Following the open-pored hydrophilic foam material 8 a plasma production device 9 is arranged in the direction of flow that provides a dielectrically obstructed discharge and thus produces a plasma that results in the removal of gaseous organic carbon compounds.

In order to limit the space requirements as much as possible, the air stream to be cleaned then is deflected by means of a plurality of fans 10 arranged side by side and subsequently arranged curved guiding surfaces 11 to flow in the opposite direction.

The air stream to be cleaned then is guided to an electric filter 12 with an ionization device 13 and collector surfaces 14.

This is followed, in the direction of flow, by a plurality of cartridges 15 that are filled with active carbon 16 or a similarly suitable absorption material.

The air stream then exits again through suitable openings on an upper side 17 of the cleaning device 1.

In a different exemplary embodiment of a cleaning device 1 according to FIG. 2 a heat exchanger 18 is arranged in the area of an intake opening 2 without any stream guide impingement baffles which causes the cooling of the air stream flowing through the intake opening 2 instead of the spraying device 5 shown in FIG. 1.

Instead of the active carbon 16 filled cartridges 15 shown in FIG. 1, the air stream in the cleaning device 1 according to FIG. 2 is guided through a mat 19 of active carbon prior to exiting the cleaning device 1.

1. Method for cleaning a hot air stream, wherein the air stream is guided through a cooling device and then through a filter device, which comprises at least one plasma production device for removing gaseous organic carbon compounds.

2. Method according to claim 1 wherein the hot air stream is cooled to temperatures below 60° C. by means of the cooling device before the cooled air stream then is guided to the filter device.

3. Method according to claim 1 wherein the air stream to be cleaned is accelerated before entering the filter device.

4. Method according to claim 1 wherein the air stream to be cleaned is deflected by means of suitable guiding surfaces.

5. Cleaning device for a hot air stream with a filter device that comprises at least one plasma production device for removing gaseous organic carbon compounds and comprises a cooling device upstream from the filter device.

6. Cleaning device according to claim 5 wherein the cooling device comprises a spraying device for a coolant.

7. Cleaning device according to claim 5 wherein the filter device comprises an open-pored hydrophilic foam material, the plasma production device and a suitable absorber material through which the air stream flows consecutively.

8. Cleaning device according to claim 5 wherein the filter device additionally comprises an electric filter.

9. Cleaning device according to claim 5 wherein the cleaning device comprises an air intake with a narrowing and subsequently widening intake opening.

10. Cleaning device according to claim 5 wherein the cleaning device comprises a plurality of fans arranged side by side.

11. Cleaning device according to claim 5 wherein a plurality of guiding surfaces are arranged side by side in the direction of flow.
